(19) United States
(12) Patent Application Publication
(10) Pub. No.: US 2006/0269963 A1
(43) Pub. Date: Nov. 30, 2006

(54) Composition for the diagnosis of retinal vascular disease comprising aldolase and method for diagnosis using it

(76) Inventors: Yang-Je Cho, Seoul (KR); Bo-Young Ahn, Seoul (KR); Won-Il Yoo, Gyeonggi-do (KR); Oh-Woong Kwon, Gyeonggi-do (KR)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 10/555,025

(22) PCT Filed: Mar. 14, 2005
(86) PCT No.: PCT/KR05/00722

§ 371 Date: Feb. 21, 2006

(30) Foreign Application Priority Data
Jul. 8, 2004 (KR) 10-2004-0052885

(51) Int. Cl. G01N 33/53; C12Q 1/26

(52) U.S. Cl. 435/7.1; 435/25

(57) ABSTRACT

Disclosed is a composition comprising an aldolase protein for diagnosing retinal vascular disease. Also, the present invention discloses a kit comprising the protein for diagnosing retinal vascular disease, and a method for diagnosing retinal vascular disease comprising bringing a blood sample into contact with the aldolase protein and quantitatively analyzing formed antigen-antibody complexes.

### TECHNICAL FIELD

The present invention relates to a composition comprising an aldolase protein for diagnosing retinal vascular disease, a kit comprising the said protein, and a method for diagnosing retinal vascular disease comprising bringing a blood sample into contact with the aldolase protein and quantitatively analyzing the formed antigen-antibody complexes.

### BACKGROUND ART

Diabetes mellitus is a complex metabolic disease that causes lesions in the microvascular system. The disease brings about a wide range of disorders in systemic tissues, and is the most important systemic disease that particularly affects the eyes (T H Lee and Y G Choi, Diabetic Vascular Complications, 1993, Korea Medical Book Publisher, Seoul, Korea). Among them, diabetic retinopathy is one of the most severe complications and is becoming an increasingly important social problem as life expectancy increases due to improved living standards and advances in medical technology (Klein R. et al., Arch Ophthalmol., 102, 520-532, 1984). There are two types of diabetic retinopathy: non-proliferative diabetic retinopathy (NPDR) in which lesions in the retina, caused by vascular disorders, are within the retina; and proliferative diabetic retinopathy (PDR) in which new vessels growing on the retina penetrate the vitreous (Green, In: Spencer W H, ed., Ophthalmic Pathology: an atlas and textbook. 4th ed., Philadelphia: W B Saunder; 1124-1129, 1996). Diabetic retinopathy vision impairment is caused by vitreous hemorrhage and tractional retinal detachment in the macular area along with macular degeneration, and surgical and laser treatment is known to be effective (Diabetic Retinopathy Study Report Number 14, Int Ophthalmol Clin., 27, 239-253, 1987). Vision loss can be prevented with minimal side effects when this treatment is performed at proper stages. Therefore, it is important to carry out the diagnosis of diabetic retinopathy frequently in order to identify the appropriate time for a surgical operation.

Diabetic retinopathy is diagnosed by examining characteristic structural changes of the fundus by fundus photography which is usually performed in an ophthalmic hospital. Diagnosing diabetic retinopathy at an early stage in patients with diabetics who do not realize a vision abnormality or do not receive a periodic ocular examination is difficult. Consequently, diabetic patients often receive surgical treatment when the condition is too far advanced and cannot be prevented.

As such, there is a need to find a method for accurately diagnosing diabetic retinopathy in its early stages, but no proper method has been reported yet.

Based on this background, the present inventors found that autoantibodies are formed in diabetic retinopathy patients because retinal proteins get exposed to the immune system, while retinal proteins are not exposed to the immune system under normal conditions due to the blood-ocular barrier present in ocular vessels. The present inventors screened retinal proteins producing autoantibodies, and found that a retinal vascular disease could be diagnosed with a high reliability by detecting an autoantibody to the aldolase protein in the patient.

### DISCLOSURE OF THE INVENTION

It is therefore an object of the present invention to provide a composition for diagnosing a retinal vascular disease, comprising an aldolase protein.

It is another object of the present invention to provide a diagnostic kit for a retinal vascular disease, comprising an aldolase protein.

It is a further object of the present invention to provide a method for diagnosing a retinal vascular disease, comprising bringing a biological sample into contact with an aldolase protein and detecting formed antigen-autoantibody complexes.

### BRIEF DESCRIPTION OF THE DRAWINGS

The accompanying drawings integrated within the present specification and comprising a portion of the specification illustrate preferred embodiments of the present invention, and may function to describe the principle of the present invention along with the following detailed description of preferred embodiments.

FIG. 1 shows the results of screening serum samples obtained from normal subjects, diabetic patients, patients with non-proliferative diabetic retinopathy and patients with proliferative diabetic retinopathy, by Western blot analysis using cytosol and membrane fractions of human retinal proteins;

FIG. 2 shows the results of two-dimensional (2-D) electrophoresis of human retinal proteins;

FIG. 3a shows the results of Western blotting of a 2-D electrophoresis gel of FIG. 2, which has been cut into four pieces, using sera from healthy male subjects;

FIG. 3b shows the results of Western blotting of the 2-D electrophoresis gel of FIG. 2, which has been cut into four pieces, using sera from patients with non-proliferative diabetic retinopathy;

FIG. 3c shows the results of Western blotting of the 2-D electrophoresis gel of FIG. 2, which has been cut into four pieces, using sera from patients with proliferative diabetic retinopathy;

FIG. 4 shows the results of diagnosis of diabetic retinopathy by ELISA analysis of sera from normal subjects, diabetic patients, patients with non-proliferative diabetic retinopathy and patients with proliferative diabetic retinopathy, using creatine kinase B;

FIG. 5 shows the results of diagnosis of diabetic retinopathy by ELISA analysis of sera from normal subjects, diabetic patients, patients with non-proliferative diabetic retinopathy and patients with proliferative diabetic retinopathy, using aldolase; and

FIG. 6 shows the results of diagnosis of diabetic retinopathy by ELISA analysis of sera from patients with diabetic retinopathy treated successfully, for example, via surgical operation, and patients with progressing diabetic retinopathy, using aldolase.

### BEST MODE FOR CARRYING OUT THE INVENTION

In one aspect, the present invention provides a composition for diagnosing a retinal vascular disease, comprising an aldolase.

The term “diagnosis”, as used herein, refers to the detection of the presence or properties of pathogenic states. With respect to the objects of the present invention, “diagnosis” means to detect a retinal vascular disease.

The term “retinal vascular disease”, as used herein, refers to all diseases in which retinal proteins are exposed to ocular vessels. A retinal vascular disease, in which autoantibodies to retinal proteins are produced in blood, can be diagnosed by detecting the production of such autoantibodies. In the present invention, a retinal vascular disease is diagnosed by detecting the formation of an autoantibody to the aldolase protein. Thus, with respect to the objects of the present invention, the retinal vascular disease includes all retinal vascular diseases that produce an autoantibody to an aldolase. Non-limiting examples of retinal vascular diseases include diabetic retinopathy, age-related macular degeneration and retinal edema. The most preferred example is diabetic retinopathy. The detection of an autoantibody against aldolase C allows for effective diagnosis of both non-proliferative and proliferative diabetic retinopathy.

The term “autoantibody”, as used herein, refers to an antibody that, unlike antibodies produced against exogenous antigens in the immune system, is produced against an endogenous or native substrate. With respect to the objects of the present invention, an autoantibody indicates an autoantibody that is produced against an exposed retinal protein in a retinal vascular disease. Retinal proteins producing autoantibodies are described in Table 2, below. These autoantibodies are usually not detectable or at most are detected at negligible levels in normal individuals or diabetic patients, but increase to significant levels in retinal vascular diseases such as diabetic retinopathy.

The present inventors found that the retinal proteins listed in Table 2 are proteins that produce autoantibodies with the incidence of diseases such as diabetic retinopathy by one-dimensional and two-dimensional Western immunoblotting, and that retinal vascular diseases could be successfully diagnosed by detecting autoantibodies to these proteins.

When blood samples from diabetic patients were analyzed by ELISA using the proteins listed in Table 2. Retinal vascular diseases were able to be diagnosed significantly through the detection of an autoantibody to the aldolase C. The term “significance”, as used herein, refers to diagnosis results having high validity coming from accurate results, and high reliability supplying constant results upon repeated measurement.

An aldolase protein is used as an antigen in order to detect an autoantibody to aldolase C present in biological samples including plasma, a serum and blood.

The aldolase, used herein as an antigen for the immuno-complex with an autoantibody to aldolase C in the present invention includes aldolase A, aldolase B and aldolase C.

There are three aldolase isoenzymes, aldolase A, B and C, and these isozymes show different tissue distributions. Aldolase A is expressed mainly in muscle and red blood cells, aldolase B mainly in the liver, kidney and small intestine, and aldolase C mainly in the brain and neuronal tissues. A very high homology exists between aldolase A, B and C in amino acid sequences, and it is known to that the structures of isozymes A, B, and C are almost identical in their overall fold and active site structure (Arakaki et al., Protein Sci. 2004 December, 13(12)3077-3084). Also, aldolase is known to be highly homologous between the animal species, for example, humans, rats, mice, etc. Taking into consideration that the interaction between an antigen and an antibody in the complexes is determined by the protein structures specified by the amino acid sequences of the proteins, a persion skilled in the art will easily understand that aldolase A and B as well as aldolase C are all able to bind to an autoantibody of the aldolase C.

Therefore, an aldolase protein usable as an antigen for the autoantibody detection of the present invention may be aldolase A, aldolase B or aldolase C, which is derived from animals including humans, goats, cows, monkeys, sheep, pigs, mice, rabbits, hamsters, rats and guinea pigs, as long as it binds to the autoantibody and forms antigen-autoantibody complexes. Since autoantibodies to aldolase A and B are not formed in retinal vascular diseases, cross-reactivity is not a cause of concern when aldolase A or aldolase B is used as a detection antigen. In Examples 4 and 5, which will be described later, when aldolase (Sigma, A2714) isolated from rabbit muscle was used as an antigen for detection of the autoantibody, patients with proliferative and non-proliferative diabetic retinopathy were distinguished from normal subjects and patients with diabetes only.

In addition, the aldolase used herein as an antigen includes aldolase variants, examples of which are amino acid sequence variants. The term “amino acid sequence variant”, as used herein, means to have a sequence including one or more amino acid residues different from the native amino acid sequence, and may be naturally or artificially generated. Alteration of an amino acid sequence includes variants by deletions, insertions, conservative or non-conservative substitutions, or combinations thereof. Preferred is a variant having a homology of 70% or higher.

The term “homology”, as used herein, indicates the degree of sequence similarity in comparison with a wild-type amino acid sequence. The homology evaluation may be done manually or using a commercially available program. Using a commercially available computer program, the homology between two or more sequences may be expressed as a percentage (%). The present invention includes amino acid sequences with 70% or higher, more preferably 80% or higher and even more preferably 90% or higher homology to an amino acid sequence encoding a wild-type aldolase.

The aldolase variant is a functional equivalent that exerts the same biological activity as does the native protein, or is preferably a variant having enhanced binding affinity or binding specificity to the autoantibody.

In addition, the aldolase used as an antigen for an autoantibody to aldolase C (anti-aldolase C autoantibody) includes antigenic fragments of the aforementioned aldolase.

The term “antigenic fragment”, as used herein, refers to a fragment that contains one or more epitopes capable of specifically binding to an antigen binding site of an antibody, specifically, an anti-aldolase C autoantibody. In detail, an antigenic fragment is a fragment of aldolase A, aldolase B, aldolase C, or a variant thereof, which includes one or more epitopes. The length of the fragment is not specifically limited as long as it acts as an antigen specifically binding the autoantibody.

The aldolase may be obtained by a variety of methods widely known in the art, including extraction and purification from natural sources, chemical synthesis using a solid-phase peptide synthesis technique, and cell-free protein synthesis. Also, a genetic recombination technique may be used to isolate and purify a recombinant protein from animal cells or microorganisms. When a genetic recombination technique is used, aldolase may be obtained by inserting a nucleic acid encoding an aldolase protein into a suitable expression vector, transforming a host cell with the vector and culturing the transformant to express aldolase, and recovering expressed aldolase from the host cell. Aldolase may be isolated and purified by general biochemical isolation techniques, for example, treatment with a protein-precipitating agent (salting out), centrifugation, ultrasonication, ultrafiltration, dialysis, various chromatographic techniques, including molecular sieve chromatography (gel filtration), absorption chromatography, ion exchange chromatography and affinity chromatography. Typically, the techniques are used in combination of two or more so as to isolate a highly pure protein.

Detailed examples of the aldolase used as an antigen for an autoantibody to aldolase C according to the present invention include human aldolase A having the amino acid sequence of SEQ ID NO. 1 (GenBank NP_{—}908932, NP_{—}908930), human aldolase B having the amino acid sequence of SEQ ID NO. 2 (GenBank NP_{—}000026, CAI14615), and human aldolase C having the amino acid sequence of SEQ ID NO. 3 (GenBank AAP35652, NP_{—}00515).

In another aspect, the present invention relates to a kit for diagnosing a retinal vascular disease, comprising aldolase.

The kit, which is for diagnosing a retinal vascular disease by measuring levels of an anti-aldolase C autoantibody in a biological sample, includes an aldolase protein that serves as an antigen reacting with an anti-aldolase C autoantibody.

Antigen-antibody complex formation may be detected by immunological techniques, which are exemplified by Western blotting, enzyme-linked immunosorbent assay (ELISA), radioimmunoassay (RIA), radioimmunodiffusion, ouchterlony immunodiffusion, rocket immunoelectrophoresis, histoimmunological staining, immunoprecipitation assay, complement fixation assay, immunofluorescence, FACS and protein chips, but the present invention is not limited to these examples.

In addition to the aldolase specifically binding to the autoantibody, the present kit for diagnosing a retinal vascular disease may include tools, reagents, and the like, which are generally used in the art for immunological analysis. These tools/reagents include, but are not limited to, suitable carriers, labeling substances capable of generating detectable signals, solubilizing agents, detergents, buffering agents and stabilizing agents. When the labeling substance is an enzyme, the kit may include a substrate allowing the measurement of enzyme activity and a reaction terminator. The diagnostic kit of the present invention may be in the type of a microplate, a dip-stick device, an immunochromatography test strip, a radial partition immunoassay device, a flow-through device, etc. Also, the diagnostic kit of the present invention may include positive and negative standard controls.

Preferably, the diagnostic kit is an ELISA diagnostic kit. ELISA includes a variety of ELISA methods, including an ELISA method using a secondary labeled antibody recognizing a capture antibody forming complexes with an antigen immobilized on a solid support, and sandwich ELISA, in which a captured antigen bound to an antibody immobilized on a solid support is detected by first adding an antigen-specific antibody, and then a secondary labeled antibody which binds the antigen-specific antibody. More preferably, the antigen-antibody complex formation is detected by an ELISA method, in which a serum sample reacts with an antibody immobilized on a solid support, and the resulting antigen-antibody complexes are detected by adding a secondary labeled antibody which binds to the antigen-specific antibody, followed by enzymatic development.

The aforementioned ELISA kit may include a secondary antibody binding which binds to the autoantibody of aldolase C. The secondary antibody labeled with a detection label is preferably an anti-human immunoglobulin G or anti-human immunoglobulin M antibody. The secondary antibody acts as a detection antibody. Since the secondary antibody possesses a detection label, the amount of the autoantibody may be determined by measuring the signal size of the detection label.

The detection label may be selected from the group consisting of enzymes, fluorescent substances, ligands, luminescent substances, microparticles, redox molecules and radioactive isotopes, but the present invention is not limited to the examples. Examples of enzymes available as detection labels include, but are not limited to, β-glucuronidase, β-D-glucosidase, β-D-galactosidase, urase, peroxidase or alkaline phosphatase, acetylcholinesterase, glucose oxidase, hexokinase and GDPase, RNase, glucose oxidase and luciferase, phosphofructokinase, phosphoenolpyruvate carboxylase, aspartate aminotransferase, phosphenolpyruvate decarboxylase, and β-latamase. Examples of the fluorescent substances include, but are not limited to, fluorescin, isothiocyanate, rhodamine, phycoerythrin, phycocyanin, allophycocyanin, o-phthaldehyde and fluorescamin. Examples of the ligands include, but are not limited to, biotin derivatives. Examples of luminescent substances include, but are not limited to, acridinium microparticles include, but are not limited to, colloidal gold and colored latex. Examples of the redox molecules include, but are not limited to, ferrocene, ruthenium complexes, viologen, quinone, Ti ions, Cs ions, diimide, 1,4-benzoquinone, hydroquinone, K₄W(CN)₈, [Os(bpy)₃]²⁺, [RU(bpy)₃]²⁺, and [MO(CN)₈]⁴⁻. Examples of the radioactive isotopes include, but are not limited to, ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re.

In a further aspect, the present invention relates to a method for diagnosing retinal vascular disease, comprising bringing a biological sample into contact with an aldolase and detecting formed antigen-autoantibody complexes.

Using this method, a patient suspected of having a retinal vascular disease such as diabetic retinopathy may be diagnosed to determine whether the patient substantially has the disease by comparing levels of antigen-autoantibody complexes in a specimen from the patient with those in a control.

The biological sample in which an anti-aldolase autoantibody is detected includes, but is not limited to, blood, serum and plasma.

The term “antigen-autoantibody complexes”, as used herein, refers to binding products of an anti-aldolase C autoantibody and an aldolase antigen. The amount of antigen-antibody complexes formed may be quantitatively measured using a secondary antibody reacting with the autoantibody.

For example, the method of diagnosing a retinal vascular disease comprises the following steps:

1) bringing a biological sample into contact with the aldolase to form antigen-autoantibody complexes; 2) incubating the complexes with a secondary labeled antibody to the autoantibody; and 3) measuring signal size of the secondary labeled antibody.

Herein, a retinal vascular disease may be diagnosed by evaluating absolute (e.g., μg/ml) or relative (e.g., relative intensity of the signal) differences to determine whether there is a significant difference between a control and a sample of interest in levels of formed antigen-autoantibody complexes.

The antigen used to form antigen-autoantibody complexes may include the aforementioned aldolase isozymes, and variants or fragments thereof and anti-idiotype antibodies thereof. The term “anti-idiotype antibody”, as used herein, refers to an antibody recognizing a variable region, that is, an idiotype region of an antibody. With respect to the objects of the present invention, an anti-idiotype antibody is an antibody to an anti-aldolase C autoantibody.

A better understanding of the present invention may be obtained through the following examples which are set forth to illustrate, but are not to be construed as the limit of the present invention.

### EXAMPLE 1

### Analysis of Autoantibodies to Human Retinal Proteins Using Western Blotting

Isolation of Human Retinal Proteins

Retinas were excised from human eyes (provided by Shinchon Severance Hospital, Seoul, Korea) and rinsed in physiological saline several times. A cytosol fraction and a membrane fraction, which are separated from each other, were obtained using a ProteoPrep Universal Extraction Kit (Sigma S2813), and retinal protein concentrations of the fractions were determined using a Pierce BCA Protein Assay Kit 23227 (Pierce, USA).

Screening of Sera from Normal Subjects and Patients by Western Blot Analysis Using Retinal Proteins

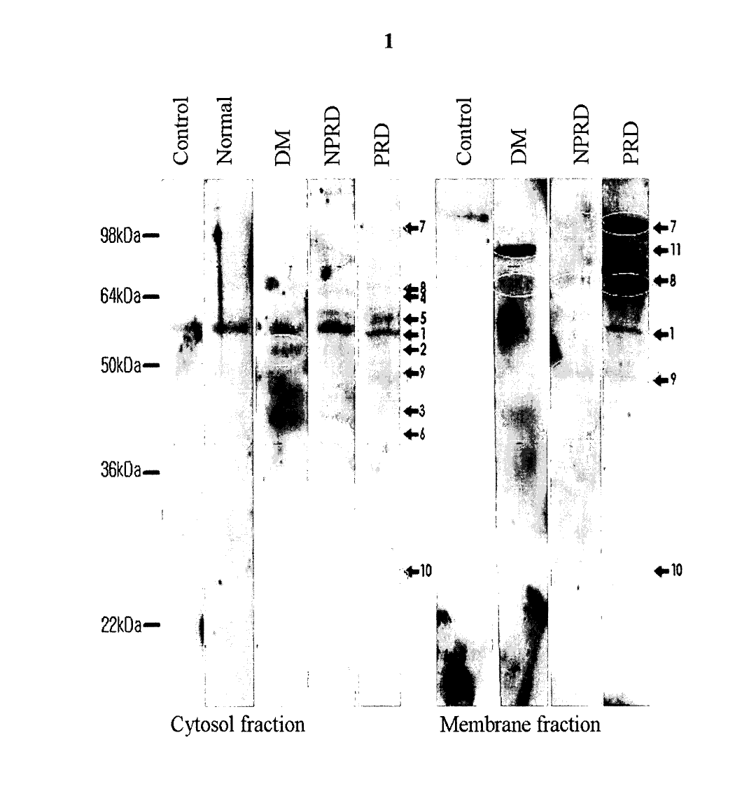

The presence of anti-retinal autoantibodies was examined by Western immunoblotting, as follows. 30 μg of the total retinal proteins was electrophoresed on a 12% acrylamide gel and transferred onto a nitrocellulose membrane. The blot was incubated with antibodies contained in serum samples (provided by Shinchon Severance Hospital, Seoul, Korea) from normal subjects, diabetic patients (DM), patients with non-proliferative diabetic retinopathy (NPDR) and patients with proliferative diabetic retinopathy (PDR), and was then incubated with an anti-human immunoglobulin G (IgG) antibody labeled with peroxidase (KOMA Biotech Inc., Korea) as a secondary antibody. The results are summarized in Table 1, below, and are also given in FIG. 1.

TABLE 1

| | | Cytosol fraction | | | | | | | | |
|---|---|---|---|---|---|---|---|---|---|---|
| IgG Heavy | MW | | Normal | | | | Membrane fraction | | | |
| chain No. | (kDa) | Control | subjects | DM | NPDR | PDR | Control | DM | NPDR | PDR |
| 1 | 56.1 | + | + | + | + | + | | | | + |
| 2 | 53.4 | | | ++ | + | + | | | | |
| 3 | 44.4 | + | + | +++ | ++ | + | | | | |
| 4 | 63.5 | | | | + | | | | | |
| 5 | 58.9 | | | | + | + | | | | |
| 6 | 40.7 | | | | | + | | | | |
| 7 | 79.2 | | | | | + | | | | + |
| 8 | 65.0 | | | | | + | | + | + | + |
| 9 | 49.6 | | | + | | ++ | | | + | + |
| 10 | 26.1 | | | | | + | | | | + |
| 11 | 73.6 | | | | | | | +++ | | |

In Table 1, “DM” indicates diabetic patients, “NPDR” indicates patients with non-proliferative diabetic retinopathy, and “PDR” indicates patients with proliferative diabetic retinopathy. The symbol “+” indicates that a positive band appears in Western blotting, and the number of the symbol indicates the intensity of the band.

### EXAMPLE 2

### 2-D Gel Electrophoresis and Western Blotting of Human Retinal Proteins

2-D Gel Electrophoresis

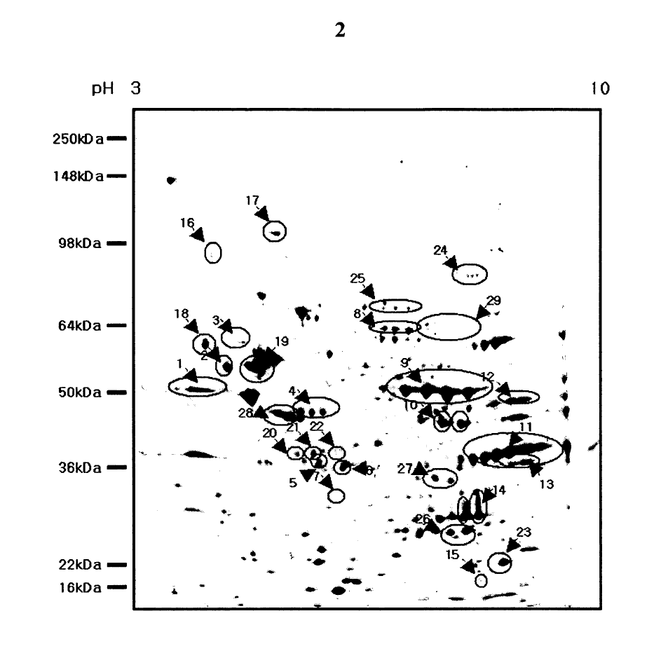

Retinal proteins were separated by two-dimensional (2-D) electrophoresis, a stepwise separation method using two different properties of proteins. Primarily, proteins were migrated according to pH (pH 3-10 gradient) by applying an electric stimulus to proteins. Secondarily, proteins were migrated on an acrylamide gel (8-18% gradient) according to molecular weights. First-dimension gel electrophoresis (protein migration according to pH) was performed with a current of 50 mA/gel for 12 hrs, and second-dimension gel electrophoresis (protein migration according to molecular weights) was performed on a polyacrylamide gel with a current of 50 mA/gel for 6 hrs. The proteins migrated thus were stained with a dye, Coomassie Brilliant Blue-250, and were also analyzed by silver staining. A total of four gels were prepared according to the procedure as described above. One of the gels was assessed for the distribution of proteins which normal subjects possess on a 2-D gel, and the results are given in FIG. 2. Numbers of FIG. 2 indicate spot numbers of Table 2, below. The remaining three gels were individually cut into four pieces and subjected to Western blotting.

Western Blotting

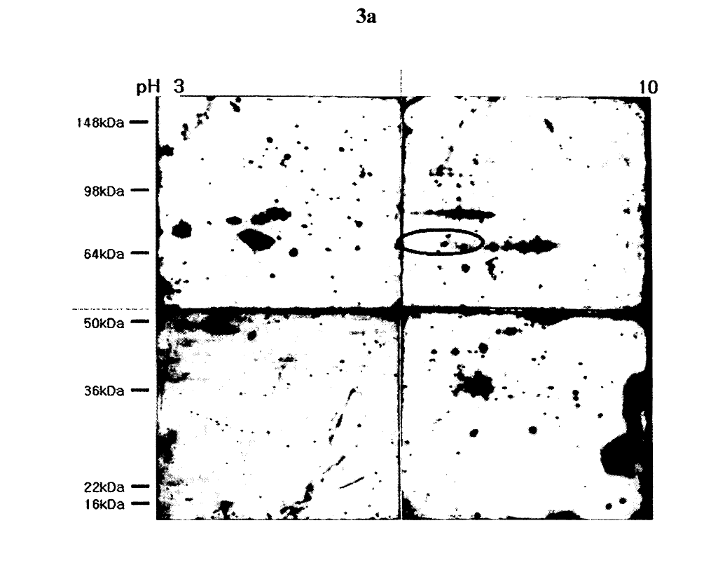

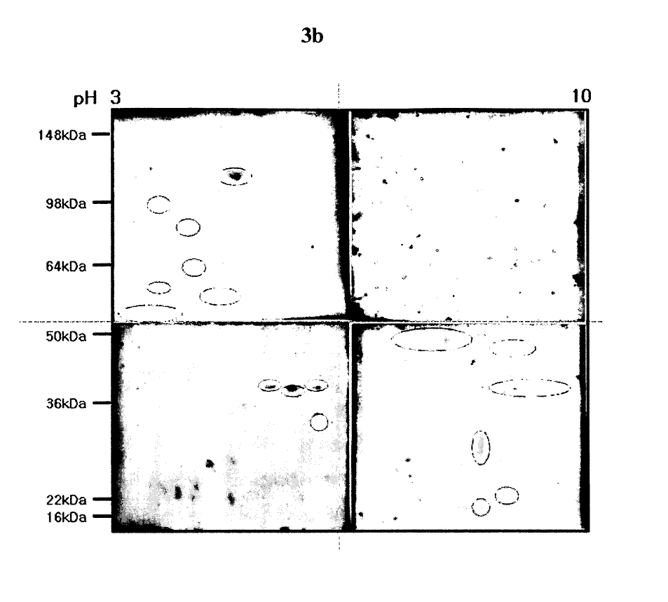

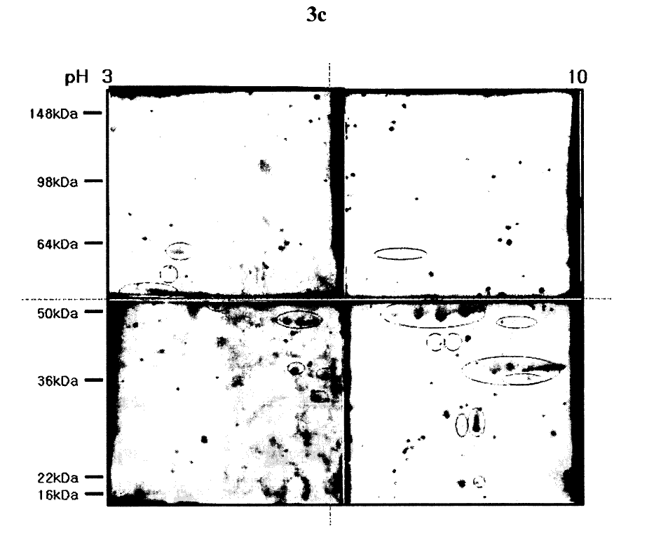

2-D electrophoresis gels were subjected to Western immunoblotting to identify anti-retinal autoantibodies. Western immunoblotting was carried out using sera of normal subjects, patients with non-proliferative diabetic retinopathy and patients with proliferative diabetic retinopathy according to the same method as in Example 1. The results are given in FIGS. 3a, 3b and 3c.

To investigate the difference in serum antibodies between normal subjects and patients with diabetic retinopathy, 2-D gel images were analyzed using image analysis software, Phoretix (Nonlinear dynamics, Great Britain). The spots, thus obtained from the 2-D electrophoresis gels through the comparison of the two groups, were analyzed by MALDI-TOF mass spectroscopy. As a result, autoantibodies against retinal proteins listed in Table 2, below, were found to exist in the sera of patients with diabetic retinopathy. Antigenic proteins for autoantibodies occurring in patients with diabetic retinopathy are summarized in Table 2, below.

TABLE 2

| Spot No. | Name of proteins |
|---|---|
| 1 | Alpha enolase (non-neural enolase) |
| 2 | Protein KIAA0193 |
| 3 | Unnamed protein product thyroid hormone binding |
| | protein precursor |
| 4 | Creatine kinase-B |
| 5 | DDAH1 protein |
| 6 | Lactate dehydrogenase B |
| 7 | Capping protein(actin filament) muscle z-line, |
| | beta |
| 8 | Dihydropyrimidinase-like 2 |
| 9 | 2-phosphopyruvate-hydratase alpha-enolase |
| 10 | Aldolase C |
| 11 | Glyceraldehyde-3-phosphate dehydrogenase |
| 12 | Phosphoglycerate kinase 1 (primer recognition |
| | protein 2 (PRP2)) |
| 13 | Lactate dehydrogenase A |
| 14 | Carbonic anhydrase II |
| 15 | Glucosidase II beta subunit |
| 16 | HS24/P52 |
| 17 | Calreticulin |
| 18 | Tubulin beta-4q chain |
| 19 | beta-tubulin |
| 20 | Guanine nucleotide-binding protein, beta-4 |
| 21 | Guanine nucleotide-binding protein(G protein), beta |
| | polypeptide 1 |
| 22 | Prostatic binding protein; phosphatidylethanolamine |
| | binding protein |

### EXAMPLE 3

### Diagnosis of Diabetic Retinopathy by ELISA Using Creatine Kinase B

An ELISA method using creatine kinase B was examined to determine whether it effectively distinguishes sera from patients with diabetic retinopathy from those of normal subjects and patients with diabetes only.

ELISA was carried out using sera (provided by Shinchon Severance Hospital, Seoul, Korea) from three normal subjects, ten diabetic patients without diabetic retinopathy and twenty patients with diabetic retinopathy.

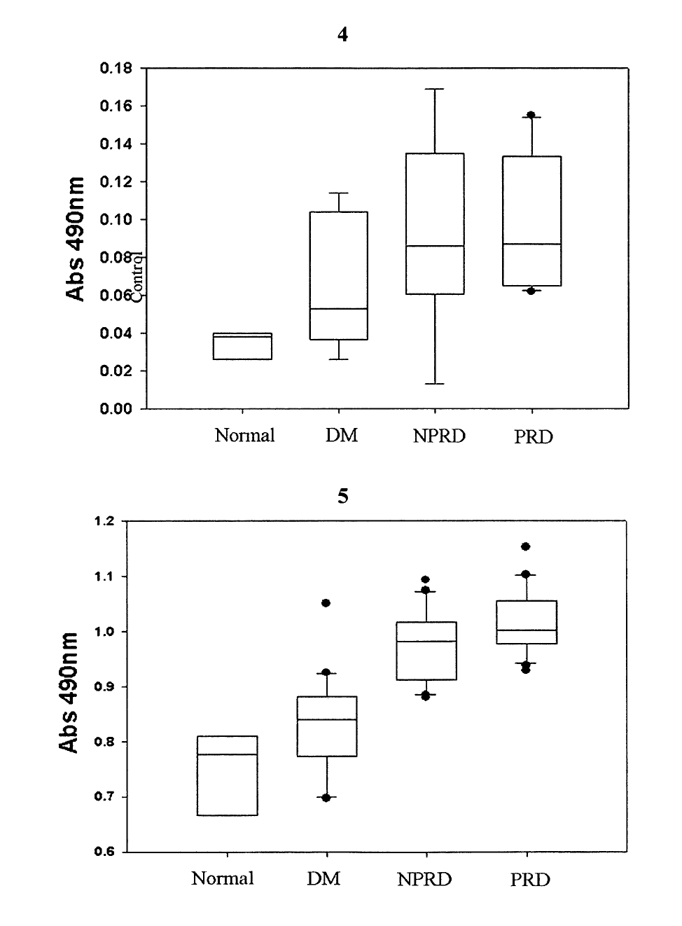

First, each well of a 96-well EIA plate was coated with 100 μl (1 μg protein per well) of creatine kinase B Sigma, C6638), dissolved in coating buffer (50 mM NaHCO₃, pH 9.0) at 10 μg/ml, at room temperature for 1 hr. After each well of the plate was washed with 400 μl of PBST (phospate buffer saline, 0.05% Tween 20) twice for 10 min for each washing, it was treated with a blocking solution, 1% BSA (bovine serum albumin) in PBS. Then, 100 μl of a patient's serum, diluted with PBST, was added to each well, followed by a 1-hr incubation. After being washed with PBS five times, each well was reacted for 1 hr with 100 μl of a dilution of an anti-human IgG antibody labeled with peroxidase (KOMA Biotech Inc., Korea). After being washed with PBS three times, each well was reacted with 100 μl of 0.1 M citrate-phosphate buffer (pH 4.9) containing 1 mg/ml OPD (o-phenylenediamine dihydrochloride) and 0.03% H₂O₂ at room temperature for 30 min. The reaction was terminated with 100 μl of 3 M sulfuric acid. Absorbance was measured at 450 nm using an ELISA reader. The results are given in FIG. 4.

As a result, mean absorbance values were 0.04 for normal subjects, 0.05 for patients with diabetes only, 0.08 for patients with non-proliferative diabetic retinopathy, and 0.08 for patients with proliferative diabetic retinopathy.

The levels of anti-creatine kinase B autoantibodies were higher in sera of patients with non-proliferative and proliferative diabetic retinopathy than in those of normal subjects and patients with diabetes only, and anti-creatine kinase B autoantibodies were effectively detected using creatine kinase B as an antigen.

### EXAMPLE 4

### Diagnosis of Diabetic Retinopathy by ELISA Using Aldolase

Diabetic retinopathy was diagnosed by detecting autoantibodies to aldolase C using ELISA employing aldolase as an antigen. The aldolase antigen used in this example is available on the market, and is derived from rabbit muscle.

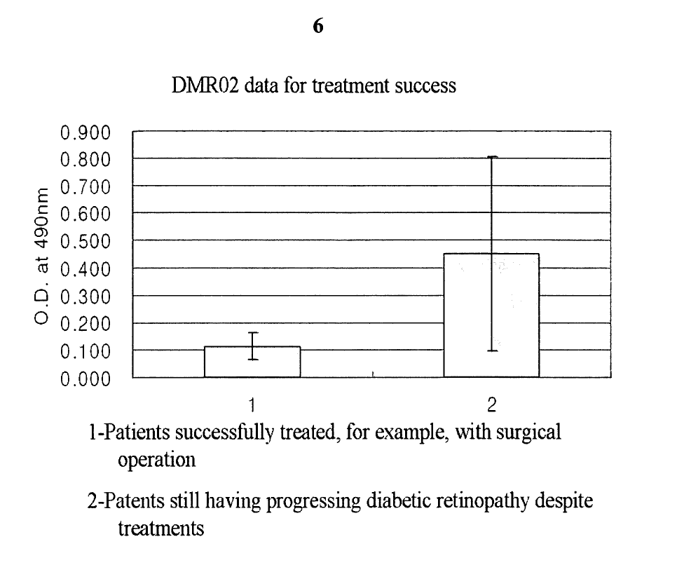

ELISA was carried out using sera (provided by Shinchon Severance Hospital, Seoul, Korea) from three normal subjects, ten diabetic patients without diabetic retinopathy and twenty patients with diabetic retinopathy. First, each well of an EIA 96-well plate was coated with 100 μl (1 μg protein per well) of aldolase (Sigma, A2714), dissolved in coating buffer (50 mM NaHCO₃, pH 9.0) at 10 μg/ml, at room temperature for 1 hr. After each well of the plate was washed with 400 μl of PBST twice for 10 min for each washing, it was treated with a blocking solution, 1% BSA in PBS. Then, 100 μl of a patient's serum, diluted with PBST, was added to each well, followed by a 1-hr incubation. After being washed with PBS five times, each well was reacted for 1 hr with 100 μl of a dilution of an anti-human IgG antibody labeled with peroxidase (KOMA Biotech Inc., Korea). After being washed with PBS three times, each well was reacted with 100 μl of 0.1 M citrate-phosphate buffer (pH 4.9) containing 1 mg/ml OPD and 0.03% H₂O₂ at room temperature for 30 min. The reaction was terminated with 100 μl of 3 M sulfuric acid. Absorbance was measured at 450 nm using an ELISA reader. The results are given in FIG. 5.

As a result, mean absorbance values were 0.78 for normal subjects, 0.84 for patients with diabetes only, 0.98 for patients with non-proliferative diabetic retinopathy, and 1.0 for patients with proliferative diabetic retinopathy. Compared to serum samples from patients with diabetes only and normal subjects using sera of normal subjects as a blank, serum samples from patients with diabetic retinophathy (proliferative and non-proliferative) displayed an increased absorbance difference of greater than about 3.

These results demonstrated that diabetic retinopathy can be diagnosed by detecting an increase in the serum level of autoantibodies against aldolase C using aldolase as an antigen.

### EXAMPLE 5

### Assessment of Diabetic Retinopathy after Treatment by ELISA Using Aldolase

Post-treatment results of patients with diabetic retinopathy, having received treatments such as surgical operations, were assessed by ELISA. ELISA was carried out using sera (provided by Shinchon Severance Hospital, Seoul, Korea) from six patients with progressing diabetic retinopathy and eleven patients having diabetic retinopathy treated, for example, via surgical operations.

ELISA was carried out according to the same method as in Example 4. Absorbance was measured at 450 nm, and the results are given in FIG. 6. As a result, mean absorbance values were 0.112 for diabetic retinopathy patients successfully treated, for example, with surgical operations, and 0.451 for patients in which diabetic retinopathy was still progressing despite treatments, thus displaying an absorbance difference greater than about 3.

The serum levels of anti-aldolase C autoantibodies were found to decrease in the successfully treated diabetic retinopathy patients. These results indicate that the detection of autoantibodies against aldolase C leads to effective analysis of post-treatment results of patients with diabetic retinopathy.

Since the examples disclosed in the present specification and constitution shown in the accompanying drawings do not represent the entire technical spirit of the present invention but are only the most preferable embodiment of the present invention, those skilled in the art will appreciate that various equivalents and modifications, capable of replacing them, are possible at the time of application of the present invention.

### INDUSTRIAL APPLICABILITY

A composition for diagnosing retinal vascular disease, a kit comprising the same and an analysis method using the same, according to the present invention allow simple and rapid diagnosis of retinal vascular diseases. Further, since the present method uses an immunological technique, it provides excellent accuracy and precision and is very cost-effective in comparison with conventional test methods.

1. A composition for diagnosing retinal vascular disease, comprising an aldolase.

2. The composition according to claim 1, wherein the aldolase is aldolase A, aldolase B, aldolase C, a variant 70% homologous thereto, or antigenic fragments thereof.

3. The composition according to claim 1, wherein the retinal vascular disease is selected from the group consisting of diabetic retinopathy, retinal edema and age-related macular degeneration.

4. A kit for diagnosing retinal vascular disease, comprising an aldolase.

5. The kit according to claim 4, wherein the aldolase is aldolase A, aldolase B, aldolase C, a variant 70% homologous thereto, or an antigenic fragment thereof.

6. The kit according to claim 5, further comprising a labeled anti-human immunoglobulin G or M antibody protein.

7. A method for diagnosing retinal vascular disease, comprising bringing a biological sample into contact with an aldolase and detecting formed antigen-autoantibody complexes.

8. The method according to claim 7, wherein the aldolase is aldolase A, aldolase B, aldolase C, a variant 70% homologous thereto, or an antigenic fragment thereof.

9. The method according to claim 7, wherein the biological sample is blood, plasma or a serum.

10. The method according to claim 7, further comprising adding a labeled anti-human immunoglobulin G or M antibody protein.


SEQUENCE LISTING (3 sequences)

SEQ ID NO: 1 - protein, 364 aa, Homo sapiens
MPYQYPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHIYLEGTLLKPNMVTPGHACTQKFSHEEIAMATVTALRRTVPPAVTGITFLSGGQSEEEASINLNAINKCPLLKPWALTFSYGRALQASALKAWGGKKENLKAAQEEYVKRALANSLACQGKYTPSGQAGAAASESLFVSNHAY

SEQ ID NO: 2 - protein, 364 aa, Homo sapiens
MAHRFPALTQEQKKELSEIAQSIVANGKGILAADESVGTMGNRLQRIKVENTEENRRQFREILFSVDSSINQSIGGVILFHETLYQKDSQGKLFRNILKEKGIVVGIKLDQGGAPLAGTNKETTIQGLDGLSERCAQYKKDGVDFGKWRAVLRIADQCPSSLAIQENANALARYASICQQNGLVPIVEPEVIPDGDHDLEHCQYVTEKVLAAVYKALNDHHVYLEGTLLKPNMVTAGHACTKKYTPEQVAMATVTALHRTVPAAVPGICFLSGGMSEEDATLNLNAINLCPLPKPWKLSFSYGRALQASALAAWGGKAANKEATQEAFMKRAMANCQAAKGQYVHTGSSGAASTQSLFTACYTY

SEQ ID NO: 3 - protein, 364 aa, Homo sapiens
MPHSYPALSAEQKKELSDIALRIVAPGKGILAADESVGSMAKRLSQIGVENTEENRRLYRQVLFSADDRVKKCIGGVIFFHETLYQKDDNGVPFVRTIQDKGIVVGIKVDKGVVPLAGTDGETTTQGLDGLSERCAQYKKDGADFAKWRCVLKISERTPSALAILENANVLARYASICQQNGIVPIVEPEILPDGDHDLKRCQYVTEKVLAAVYKALSDHHVYLEGTLLKPNMVTPGHACPIKYTPEEIAMATVTALRRTVPPAVPGVTFLSGGQSEEEASFNLNAINRCPLPRPWALTFSYGRALQASALNAWRGQRDNAGAATEEFIKRAEVNGLAAQGKYEGSGEDGGAAAQSLYIANHAY